(19) United States
(12) Patent Application Publication
(10) Pub. No.: US 2002/0137104 A1
(43) Pub. Date: Sep. 26, 2002

(54) High throughput determination of antigen expression

(76) Inventor: Antonio Cosma, Munich (DE)

Correspondence Address: BAKER & BOTTS, 30 ROCKEFELLER PLAZA, NEW YORK, NY, 10112

(21) Appl. No.: 09/994,211

(22) Filed: Nov. 26, 2001

Related U.S. Application Data
(60) Provisional application No. 60/253,706, filed on Nov. 27, 2000.

(51) Int. Cl. G01N 33/53; G01N 33/542; G01N 33/537; G01N 33/543

(52) U.S. Cl. 435/7.9; 435/7.92

(57) ABSTRACT

The present invention relates to methods and kits for high throughput determination of antigen expression in a sample, wherein a population of antigens, which have been pre-selected and then detectably labeled, are screened against an array of defined antibodies. The methods of the invention allow for the simultaneous analysis of a multitude of antigens, wherein the pre-selection and labeling of the antigens confers increased sensitivity which permits the detection of antigens which were hitherto difficult to detect using conventional labeling methods.

This application claims priority to provisional United States application No. 60/253,706 which was filed on Nov. 27, 2000.

### 1. INTRODUCTION

The present invention relates to methods and kits for high throughput determination of antigen expression in a sample, wherein a population of antigens, which has been pre-selected and then detectably labeled, is screened against an array of defined antibodies. The methods of the invention allow for the simultaneous analysis of a multitude of antigens, wherein the pre-selection and labeling of the antigens confers increased sensitivity which permits the detection of antigens which were hitherto difficult to detect using conventional labeling methods.

### 2. BACKGROUND OF THE INVENTION

As scientists attain a clearer understanding of the events surrounding cell differentiation and senescence, and of the molecular bases for diseases, numerous antigens and antigenic domains of importance have been identified. The ability to generate antibodies toward these antigens and domains has produced an arsenal of immunoglobulins which may be used to detect and quantify their targets. These molecules may be utilized by researchers to advance our comprehension of cell biology and by the medical profession to diagnose and assess disease. For example, the development of various antibodies directed toward proteins involved in the form of programmed cell death known as “apoptosis” may be used to evaluate whether various degenerative diseases, such as Alzheimer's disease or Parkinson's disease, have an apoptosis-associated etiology. In clinical applications, the pathogen associated with an undiagnosed infection may be identified by screening a patient sample with a panel of antibodies directed toward a variety of viruses, bacteria, fungi and protozoa. Oncologists have discovered associations between the expression of particular antigens on cancer cells which has led to numerous subclassifications of certain cancers, such as non-Hodgkins lymphoma, which carry substantially different prognoses. Science has amassed a sufficient number of molecular tools to be able to perform complex analyses.

Accordingly, there is a growing need to be able to screen for the presence of particular antigens. Current techniques include, among others, immunohistochemistry, flow cytometry, and ELISA analyses. These techniques suffer from a variety of shortcomings, including limitations in their ability to screen for the presence of a multitude of antigens in a short period of time. The present invention addresses this need by providing methods using microarrays of defined antibodies to simultaneously screen for the presence of a plurality of antigens. Whereas International Application No. PCT/US99/02442 (International Publication No. WO 99/40434 by Invitrogen, Hoeffler et al. inventors) relates to the use of microarrays of antibodies to analyze proteins, it fails to teach preselection and labeling of classes of proteins, an aspect of the present invention which offers the advantages of efficiency and sensitivity. Whereas U.S. patent application Ser. No. 09/146,587, U.S. Pat. No. 6,150,123 to Cosma, incorporated by reference herein, teaches pre-selection and labeling of proteins, it does not disclose the use of such methods in the context of microarrays for high throughput antigen screening, where the ability to detect antigens expressed at low levels is particularly important.

### 3. SUMMARY OF THE INVENTION

The present invention relates to methods and kits for detecting the expression of a plurality of antigens in a sample, wherein a population of antigens, which has been pre-selected and then detectably labeled, is screened against an array of defined antibodies. The pre-selection and labeling of the antigens allows the analysis of expression of different antigens selected on the basis of their chemical properties (e.g., glycoproteins, phosphoproteins, sugars, lipids, etc.) by providing for a simple system for antigen enrichment and labeling. Pre-selection confers increased sensitivity which permits the detection of antigens which were hitherto difficult to detect using conventional labeling methods. Use of microarrays allows the simultaneous analysis of potentially more than 10,000 antigens in a single analytical run.

### 4. DETAILED DESCRIPTION OF THE INVENTION

The present invention provides for methods for detecting and/or quantitating the expression of one or more of a plurality of antigens in a sample, wherein a population of antigens, which has been pre-selected and then detectably labeled, is screened against an array of defined antibodies. The present invention further provides for kits for performing such methods.

An “antigen” is defined as any molecule toward which an antibody can be generated, including molecules which require the presence of an adjuvant or conjugation to another molecule to induce antibody formation. An antigen may be a peptide or protein, a carbohydrate, a nucleic acid, or a lipid. The term “antigen” also includes portions of naturally occurring molecules, such as, for example, a subunit of a protein, or a fragment of a protein containing a functional domain or biologically relevant motif. Suitable antigens include but are not limited to proteins associated with the cell cycle, tissue-specific proteins, tumor markers, cytokines, major histocompatability complex proteins, heat shock proteins, and pathogen-associated proteins (where a pathogen may be a bacterium, a virus, a fungus, a protozoan, a multicellular parasite, or a prion), as well as carbohydrate residues associated with blood transfusion antigens and tissue typing, lipopolysaccharides, sphingolipids, etc.

A “sample”, as the term is used herein, is a sample obtained from a cell, a population of cells, a tissue, or an organism. For example, where the sample is obtained from an organism (alternately referred to herein as a “subject”), the sample may be comprised of cells, tissue, blood, serum, plasma, cerebrospinal fluid, semen, pleural fluid, peritoneal fluid, urine, feces, tears, sputum, etc.. The subject may be a human or a non-human subject.

A “population of antigens” is prepared from the sample by forming a cell-free extract (in cases where the sample is not already a cell-free extract) and first pre-selecting and then labeling a population of interest from the extract. A detailed description of selection and labeling is provided in U.S. patent application Ser. No. 09/146,587, U.S. Pat. No. 6,150,123 to Cosma, incorporated by reference herein. An extract may be formed, for example, by treating a cell with a detergent (e.g., NP40), by sonication, by freeze-thawing, or by any other method known in the art.

The “population of antigens” is selected by an affinity-based method, e.g., affinity chromatography, which selects for the class of antigen desired. The term “affinity-based method” refers to the use of a ligand which binds to the class of antigen desired bound to a solid phase element. Suitable solid phase elements include, but are not limited to, arylamide derivatives, methacrylate derivatives, polystyrene and polystyrene derivatives, magnetic beads, agarose and Sepharose™ 4B. The solid phase element may be a particle, such as a bead, or may be a surface such as a biochip or microcantilever (such as, for example but not by way of limitation, a LabChip® by Caliper Technologies Corp., a partner of Agilent Technologies; see also Xiao et al., 2001, Cancer Res. 61(16):6029-6033; Bashir et al., 2001, Biotechnol. Bioeng. 73(4):324-328; Askari et al., 2001, Biotechnol. Prog. 17(3):543-552; Wu et al., 2001, Nature Biotechnol. 19(9 :856-860). In preferred embodiments, the solid phase is a Sepharose™ bead. Where a bead is used, the ligand-bound beads may be comprised in a chromatography column or may be used as a slurry.

The type of ligand bound to the solid support depends upon the class of antigen to be selected. Where the class of antigen is glycoproteins, the ligand may be a lectin. Where the class of antigen is a type of enzyme, the ligand may be a substrate for the enzyme or an analog thereof. Where the class of antigen is defined by the presence of a particular domain, or by a structure and its homologs, the ligand may be an antibody or antibodies (monoclonal or polyclonal) which recognize the domain or structure. In other embodiments, the ligand may be a cellular receptor, a cofactor, a complementary subunit, and so forth.

The ligand may be coupled to the solid phase element by any method known in the art. Where the ligand is an antibody, the antibody may be bound to the solid phase element by, for example, a cyanogen bromide activation method, as described in “Short Protocols in Molecular Biology”, 1995, Ausubel et al., eds. John Wiley & Sons, New York, pp. 10-72-10-73.

The population of antigens is selected under conditions which favor binding of the ligand to the antigen. For example, where the affinity element is a lectin, glycoproteins may be bound by incubating a larger population of proteins with lectin bound to a solid phase element in a binding buffer which is 0.25 percent NP40 in phosphate buffered saline (“PBS”) for about three hours at 4° C, with rotation. Where the affinity element is an antibody, a method such as that described in “Short Protocols in Molecular Biology”, 1995, Ausubel et al., eds. John Wiley & Sons, New York, pp. 10-55 - 10-58 may be used. After binding, the solid phase may be washed with an effective volume of a solution (for example, a volume at least 5-10 times the volume of the solid phase) to remove unbound sample components.

The selected antigens, bound to the solid phase element via the ligand, may then be detectably labeled. The label may be, for example but not by way of limitation, biotin, a radioisotope, a fluorescent or potentially fluorescent compound, or an epitope. The label may be detected directly (e.g. a radioisotope) or by binding to one or more additional molecules (e.g., an epitope which binds to a labeled antibody). Non-limiting examples of fluorescent compounds which may be used according to the invention include fluorochromes such as Fluorescin or Alexa 488, Cy 3, or Cy 5 (which have emission maxima at 520 nm, 565 nm, and 667 nm respectively) and green fluorescent protein. After labeling, the selected proteins may be removed from the solid phase element by, for example, competitive, non-denaturing methods (e.g. using excess ligand or ligand analog; altering ionic strength or pH).

Where labeling is performed using biotin, biotinylation may be performed, for example, by reaction with NHS-biotin (see below) at a concentration of about 3.2 percent (volume/volume) for 30 minutes at 4° C., or using any method known in the art, for example, as set forth in Meier et al., 1992, Anal. Biochem. 204:220-226 and Nesbitt and Horton, 1992, Anal. Biochem. 206:267-272. Unreacted biotin may be removed by washing the solid phase and its retained protein with an effective volume of a solution, typically at least 10-20 times the volume of the solid phase.

In a specific, non-limiting example, lectin from Lens culinaris coupled to Sepharose™ 4B (Seph-LcH) may be purchased (e.g. from Sigma Chemical Co., St. Louis, Mo.). To provide the desired ratio, 200 microliters of cell lysate may be combined with 75 microliters of Seph-LcH in 150 microliters of 0.25 percent NP40 in PBS for 3 hours at 4° C., with rotation. The resulting Seph-LcH/glycoprotein complex may be washed with at least 10 bed volumes of PBS/0.25% NP40 to remove unbound sample components, and then resuspended 1:4 with the same buffer before addition of biotin-spacer arm-N-hydroxysuccimide ester in dimethylformamide (Amersham, Little Chalfont, UK;″NHS-biotin; 3.2 percent volume/volume). The Seph-LcH-glycoprotein complex was then incubated with NHS-biotin for 30 minutes at 4° C. with gentle rotation, and then successively washed with 20 bed volumes of PBS/0.25% NP40 to remove unreacted biotin. After labeling, the glycoproteins may be eluted from the Seph-LcH by incubation with 400 microliters of PBS/0.25% NP40 buffer containing the competitive inhibitor methyl α-D-mannopyranoside (1M) for 2 hours at 4° C. with gentle rotation.

Selected, labeled proteins may then be analyzed for binding with one or more of a microarray of defined antibodies bound to a solid support. The term “defined antibodies” includes within its scope monoclonal antibodies for which a target antigen has been identified as well as monoclonal or polyclonal antibodies defined by the antigen used to induce their formation (e.g., monoclonal or polyclonal antibodies generated by immunization with a particular protein domain). The solid support may be any surface to which an antibody may be covalently or non-covalently bound. In preferred non-limiting embodiments of the invention, the solid support is a glass slide and the antibodies are adhered by essentially the procedure set forth in MacBeath and Schreiber, 2000, Science 289:1760-1763; see also Askari et al., 2001, Biotechnol. Prog. 17(3):543-552 which describes an “antibody biochip”. The term “microarray” as used herein refers to a plurality of antibodies arranged on the solid support such that the position of a particular antibody on the solid support is defined. For example, antibodies may be deposited as a series of spots across a glass slide, where the identity of the antibody associated with each spot is known. Antibodies may be deposited on the solid support, for example, manually or robotically.

Binding of antigen to antibody may be detected and/or quantified using methods known in the art and dependent on the nature of the label used. For example, where the label is a fluorochrome, antigen binding may be detected by laser excitation of the fluorochrome, which yields an emission with a characteristic spectrum measurable by a fluorescent slide scanner. Preferably, in each analytical run one or more reference (control) plus one test sample are tested on the same array using fluorochromes having different emission spectra. If antigens are labeled with biotin or epitope tag, for example, a secondary reagent would be necessary to detect antigen binding (e.g. Streptavidin coupled to fluorochrome, epitope-binding antibody conjugated to fluorochrome). Analysis of each analytical run may be performed by comparing the signal obtained with the reference sample with that of the test sample.

One specific non-limiting embodiment of the invention is a method for measuring the levels of cytokines produced by lymphocytes of a subject. Cytokines are classified as TH1 or TH2 on the basis of their cell origin and biological functions. For example, TH1 cytokines such as interleukin-2 (“IL-2”), gamma interferon (“γIFN”), interleukin-12 (“IL-12”) support cell-mediated immunity and DTH-type responses, whereas TH2 cytokines such as interleukin-4 (“IL-4”), tumor necrosis factor beta (“TNF-β) and interleukin-10 (“IL-10”) support antibody-mediated immunity. Different diseases are characterized by a TH1 versus TH2 polarization of T cell responses; e.g., in human immunodeficiency virus (“HIV”) infection a switch toward TH2 responses is associated with disease progression.

According to the present invention, antibodies specific for a variety of TH1 and TH2 cytokines may be use to analyze the cytokine profile of a subject suffering from HIV infection and thereby assess the clinical status of the subject. For example, peripheral blood lymphomonocytes may be collected from two healthy control individuals as well as the subject infected with HIV. The cells are harvested using standard techniques from a ficoll Hypaque preparation. The sample from the HIV-infected subject (hereafter referred to as the “patient sample”) is lysed with a solution of 0.5 percent NP40 in phosphate buffered saline (“PBS”) in the presence of a protease inhibitor. To produce TH1 and TH2 controls, the cells from the control subjects are stimulated in vitro using standard techniques to induce the production of TH1 or TH2 cytokines, and are referred to hereafter as “TH1-ref” and “TH2-ref” samples. Following stimulation, the cells are lysed as set forth above for the cells of the patient sample.

Cell lysates from the TH1-ref, TH2-ref, and patient samples may then be diluted two times and incubated with lectin-coupled Sepharose beads, thereby allowing for the separation of glycoproteins. The beads may then be washed and the three glycoprotein pools, bound to the beads, may be labeled, for example with three different detectable labels so as to permit simultaneous analysis of reference and patient samples. In a non-limiting example, the Sepharose-bound TH1-ref, TH2-ref, and patient sample glycoproteins may be labeled, respectively, with Alexa-488, Cy-3, and Cy-5, fluorochromes having non-overlapping 520 nm, 565 nm, and 667 nm emission maxima. After labeling, excess unreacted label may be washed away from the beads and the labeled glycoproteins may be eluted from the lectin-Sepharose beads by addition of an excess of sugar.

To analyze cytokine expression in the reference and patient samples, antibodies to various cytokines may be arrayed covalently on glass microscope slides, for example as described in MacBeath and Schreiber, 2000, Science 289:1760-1763. Briefly, slides are treated with an aldehyde-containing silane reagent, wherein the aldehyde reacts with primary amines on the proteins to form a Schiff's base linkage, with the result that the proteins are covalently linked to the glass slide by their amino termini or by lysines.

The specific antibodies may be delivered onto the aldehyde/silane-treated slide manually or, for example, by a contact printing robot, to deposit one or several nanoliters of antibody solution having a concentration of about 0.1 to 0.5 mg of antibody per milliliter. Antibody may be spotted onto the slide in a 40 percent glycerol buffer to prevent evaporation during the procedure. To permit protein linkage to the slide, the slide may be incubated for about 3 hours after deposition of antibody, after which it may be washed and then incubated with a solution of bovine serum albumin to quench remaining free aldehydes.

In a specific non-limiting example of the invention, antibodies directed toward the TH1 cytokines IL-2, IL-12, and IFNγ and toward TH2 cytokines IL-4, IL-10 and TNF-β may be deposited onto a glass slide as six discrete spots and linked to the slide by the Shiff's base linkage described above. Alexa-488, Cy-3 and Cy-5 fluorochrome-labeled glycoproteins from the TH1-ref, TH2-ref and patient samples may then simultaneously be exposed to the slide under conditions that promote antibody-antigen binding. Thereafter, the slide may be washed and analyzed by a fluorescence slide scanner to detect emissions at 520 nm (indicative of bound TH1-ref cytokine binding), 565 nm (indicative of TH2-ref cytokine binding), and 667 nm (indicative of patient sample cytokine binding). If the patient cytokine profile is of the TH1 type, the results may be as set forth in TABLE 1; if the patient cytokine profile is of the TH2 type (indicating disease progression), the results may be as set forth in TABLE 2. The results may be imported into a software where they are pseudo-colored and merged.

TABLE 1
Results For A Patient Manifesting TH1-type Cytokines

| IL-2 | IL-12 | IFNγ | IL-4 | IL-10 | TNF-β | λ/sample |
|---|---|---|---|---|---|---|
| + | + | + | − | − | − | 520 nm |
| | | | | | | TH1-ref |
| − | − | − | + | + | + | 565 nm |
| | | | | | | TH2-ref |
| + | + | + | − | − | − | 667 nm |
| | | | | | | patient |
| | | | | | | sample |

TABLE 2
Results For A Patient Manifesting TH2-type Cytokines

| IL-2 | IL-12 | IFNγ | IL-4 | IL-10 | TNF-β | λ/sample |
|---|---|---|---|---|---|---|
| + | + | + | − | − | − | 520 nm |
| | | | | | | TH1-ref |
| − | − | − | + | + | + | 565 nm |
| | | | | | | TH2-ref |
| − | − | − | + | + | + | 667 nm |
| | | | | | | patient |
| | | | | | | sample |

As an alternative to labeling with different fluorochromes and scanning a slide reacted with TH1-ref, TH2-ref, and patient sample glycoproteins simultaneously, the control and patient glycoproteins may be labeled with the same compound and then reacted and analyzed separately. For example, the reference and patient glycoproteins, bound to lectin-Sepharose beads, may be biotinylated and then eluted using procedures set forth above. Then, three glass slides, each spotted with IL-2, IL-12, IFNγ, IL-4, IL-10, and TNF-β, may be reacted separately with the three samples of labeled glycoproteins, and then analyzed for bound antigen using avidin or Streptavidin.

The present invention further provides for kits which may be used to practice the method of the invention. Such kits may comprise, for example, microarrays of antibodies bound to a solid support and/or materials to pre-select and label classes of antigens. As a non-limiting specific example, a kit used to perform the particular embodiment described above may include glass slides carrying microarrays of IL-2, IL-12, IFNγ, IL-4, IL-10, and TNF-β, lectin-coupled Sepharose, and the necessary materials for labeling glycoproteins bound to the lectin-coupled Sepharose with fluorochromes or biotin. If biotin were used, detectably labeled streptavidin may also be included.

Various references are cited herein, the contents of which are hereby incorporated by reference in their entireties.

What is claimed is:

1. A method for detecting the expression of a plurality of antigens in a sample, comprising the steps of:
(i) preselecting the antigens;
(ii) labeling the preselected antigens; and
(iii) screening the labeled, preselected antigens against an array of defined antibodies.

2. The method of claim 1, wherein a class of antigen is preselected using an affinity method whereby one or more ligands which bind to the class of antigen are bound to a solid phase element, and a preparation comprising the antigens to be preselected is exposed, under binding conditions, to the one or more ligands bound to the solid phase element.

3. The method of claim 2, wherein the preselected antigens are freed from the ligand after labeling.

4. The method of claim 2, wherein the one or more ligands are bound to a bead.

5. The method of claim 2, wherein the one or more ligands are bound to a biochip.

6. The method of claim 3, wherein the one or more ligands are bound to a bead.

7. The method of claim 3, wherein the one or more ligands are bound to a biochip.

8. The method of claim 2, wherein the ligand is an antibody.

9. The method of claim 2, wherein the ligand is a lectin.

10. The method of claim 3, wherein the ligand is an antibody.

11. The method of claim 3, wherein the ligand is a lectin.

12. The method of claim 4, wherein the ligand is an antibody.

13. The method of claim 4, wherein the ligand is a lectin.

14. The method of claim 5, wherein the ligand is an antibody.

15. The method of claim 5, wherein the ligand is a lectin.

16. The method of claim 6, wherein the ligand is an antibody.

17. The method of claim 6, wherein the ligand is a lectin.

18. The method of claim 7, wherein the ligand is an antibody.

19. The method of claim 7, wherein the ligand is a lectin.

20. The method of claim 1, wherein the array of antibodies is attached to a glass surface.

21. The method of claim 1, wherein the array of antibodies is attached to a biochip.

22. A kit for practicing a method for detecting the expression of a plurality of antigens in a sample, comprising:
(i) a means for preselecting one or more antigen;
(ii) a means for labeling the one or more preselected antigen; and
(iii) an array of defined antibodies.
